(19) United States
(12) Patent Application Publication
(10) Pub. No.: US 2016/0175334 A1
(43) Pub. Date: Jun. 23, 2016

(54) ANTIFUNGAL COMBINATION THERAPY OF TAVABOROLE AND CICLOPIROX

(71) Applicant: Gavis Pharmaceuticals, Somerset, NJ (US)

(72) Inventor: BALA CHANDRAN NAYAR, Dayton, NJ (US)

(73) Assignee: Gavis Pharmaceuticals, Somerset, NJ (US)

(21) Appl. No.: 14/573,885

(22) Filed: Dec. 17, 2014

(51) Int. Cl. A61K 31/69; A61K 45/06; A61K 9/00; A61K 47/14; A61K 47/20; A61K 31/4418; A61K 47/10

(52) U.S. Cl. CPC A61K 31/69; A61K 31/4418; A61K 45/06; A61K 47/10; A61K 47/14; A61K 47/20; A61K 9/0014

(57) ABSTRACT

A novel combination of two antifungal agents is provided. The combination contains ciclopirox and a boron-containing antifungal agent or salts thereof. The invention also provides a method of treating onychomycosis using said antifungal agent combination.

### BACKGROUND OF THE INVENTION

(a) Field of the Invention

The present invention is directed to antifungal combination therapy comprising use of two antifungal agents, ciclopirox and a boron-containing antifungal agent or salts thereof. More particularly, the invention relates to antifungal combination therapy comprising the use of ciclopirox and a boron-containing antifungal agent, particularly tavaborole or salts thereof for treating fungal infections such as onychomycosis.

(b) Description of the Related Art

Onychomycosis is a disease of the nail caused by yeast, dermatophytes, or other molds, and represents approximately 50% of all nail disorders. Toenail infection accounts for approximately 80% of onychomycosis incidence, while fingernails are affected in about 20% of the cases. Dermatophytes are the most frequent cause of nail plate invasion, particularly in toenail onychomycosis. Onychomycosis caused by a dermatophyte is termed Tinea unguium. Trichophyton rubrum is by far the most frequently isolated dermatophyte, followed by T. mentagrophytes. Distal subungual onychomycosis is the most common presentation of tinea unguium, with the main site of entry through the hyponychium (the thickened epidermis underneath the free distal end of a nail) progressing in time to involve the nail bed and the nail plate. Discoloration, onycholysis, and accumulation of subungual debris and nail plate dystrophy characterize the disease. The disease adversely affects the quality of life of its victims, with subject complaints ranging from unsightly nails and discomfort with footwear, to more serious complications including secondary bacterial infections.

In most cases, onychomycosis is associated with limited treatment options that are effective in achieving complete clearance. In addition, recurrence rates are high in the subset of treated patients who have been effectively cleared, usually with an oral antifungal agent.

Many methods are known for the treatment of fungal infections, including the oral and topical use of antibiotics (e.g., nystatin and amphotericin B), imidazole anti-fungal agents such as miconazole, clotrimazole, fluconazole, econazole and sulconazole, and non-imidazole fungal agents such as the allylamine derivatives terbinafine and naftifine, and the benzylamine butenafine. The existing topical antifungals are not associated with dangerous adverse events, as they rarely penetrate the systemic circulation and gain a significant concentration in the body.

However, onychomycosis has proven to be resistant to most treatments. Nail fungal infections reside in an area difficult to access by conventional topical treatment and anti-fungal drugs cannot readily penetrate the nail plate to reach the infection sites under the nail. Therefore, onychomycosis has traditionally been treated by oral administration of anti-fungal drugs; however, clearly this is undesirable due to the potential for side effects of such drugs, in particular those caused by the more potent anti-fungal drugs such as itraconazole and ketoconazole. An alternative method of treatment of onychomycosis is by removal of the nail before treating with a topically active anti-fungal agent; such a method of treatment is equally undesirable. Systemic antimycotic agents require prolonged use and have the potential for significant side effects. Topical agents have usually been of little benefit, primarily because of poor penetration of the anti-fungal agents into and through the nail mass.

U.S. Pat. No. 5,030,619 discloses an antifungal or antimycotic composition comprising therapeutically effective amounts of nikkomycin and echinocandin B.

U.S. Pat. No. 6,875,740 discloses an antifungal combination use of azole and polyene antifungals in combination with a lipopeptide compound antifungal agent.

U.S. Pat. No. 8,552,042 discloses a method of treating candidiasis by using a combination of an antimycotic agent and an epithelial cell or endothelial cell adhesion inhibitor such as iloprost, cicaprost, ticlopidine or clopidogrel.

International Application WO 88/06884 teaches treating nail mycosis with a pharmaceutically effective amount of a topical antimycotic such as an imidazole compound, optionally with an antiseptic such as salicylic acid.

U.S. Pat. No. 6,416,749 discloses a method of treating onychomycosis using salicylic acid and optionally in combination with retinoid compounds such as tretinoin, adapalene, manoalide, retinol, and tretinate.

There has been a noticeable absence of medical therapies available for onychomycosis. Since the introduction of topical ciclopirox (8% nail lacquer), no new effective agent has been introduced for more than ten years. Fortunately, newer agents and formulations have been under formal development. While patients might prefer a topical therapy, the efficacy and poor therapeutic outcomes achieved with ciclopirox 8% nail lacquer were not unexpected as the cure rates achieved in the clinical trials were unimpressive despite concomitant nail debridement, which was an integral part of the pivotal trials with ciclopirox 8% nail lacquer.

Ciclopirox is a known topical antifungal or antimycotic agent belonging to the chemical class of hydroxypyridones and not related to azoles or any other class of antifungal agents. It has the chemical name 6-cyclohexyl-1-hydroxy-4-methyl-2(1H)-pyridone and structure as follows:

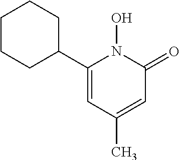

Ciclopirox 8% solution has been approved for the treatment of onychomycosis in the United States and Canada. While Ciclopirox shows a degree of efficacy against onychomycosis for certain patient populations, it has proven not to be universally effective against the illness.

U.S. Pat. No. 6,495,124 discloses a composition for the treatment of fungal infections of nails, comprising at least one antifungal agent such as ciclopirox, econazole, and a plasticizing and penetration enhancing compound.

U.S. Pat. No. 6,224,887 discloses a composition for the treatment or prevention of fungal infections of nails comprising at least one antifungal agent effective for prevention of onychomycosis selected from polyenes, allylamines, imidazoles, triazoles, ciclopirox, undecylenic acid, and amorolfine, a penetration enhancing agent, a water insoluble film forming polymer and volatile solvent.

US Application Publication No. 20100273834 discloses a composition for the treatment of onychomycosis comprising ciclopirox and urea.

US Application Publication No. 20100261695 discloses a composition for the treatment of onychomycosis comprising ciclopirox and at least one other antifungal agent. The publication discloses several conventional antifungal agents to be used in combination with ciclopirox for onychomycosis treatment.

Recently, a new class of boron containing antifungal agents have been developed. These new antifungal agents have a unique mechanism of action against fungal organisms and retain antifungal properties in the presence of keratin. Tavaborole is a boron-containing topical antifungal agent specifically developed for the treatment of dermatophyte onychomycosis. It has the chemical name of 5 fluoro-1,3-dihydro-1-hydroxy-2,1-benzoxaborole and structure as follows:

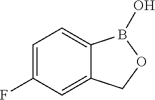 [SMILES: OB1OCc2cc(F)ccc21]

In Phase 3 clinical trials, tavaborole was applied once daily for 48 weeks without concomitant nail debridement. Mycologic cure rates with tavaborole 5% solution were markedly superior to what was achieved with ciclopirox 8% nail lacquer.

U.S. Pat. No. 7,582,621 discloses boron containing compounds including tavaborole for treating fungal infections including onychomycosis.

Despite the existence of the currently available mono and combination therapies, a new, alternate and effective drug combination for the management of onychomycosis is still required. A need for such new combination treatment options for onychomycosis also exists in the art which can effectively penetrate as well as treat the nail infection in the shortest possible duration. These and other needs are addressed by the current invention.

### SUMMARY OF THE INVENTION

The present invention generally relates to the treatment of onychomycosis. More specifically, it relates to a combination therapy of ciclopirox and a boron-containing antifungal agent or salts thereof for the treatment of onychomycosis and related conditions.

In a first aspect, the invention provides a combination of ciclopirox and a boron-containing antifungal agent or salts thereof.

In a second aspect, the invention provides a combination of ciclopirox and tavaborole or salts thereof.

In another aspect, the invention provides a pharmaceutical composition comprising (a) ciclopirox or salts thereof; (b) a boron-containing antifungal agent or salts thereof; and (c) a pharmaceutically acceptable excipient. The composition may be in a form suitable for topical administration, such as a solution, gel, cream, lotion, tincture, or ointment.

In another aspect, the invention provides a topical pharmaceutical solution consisting essentially of ciclopirox or salts thereof and a boron-containing antifungal agent or salts thereof as the antifungal agents and one or more pharmaceutically acceptable excipients to deliver the ciclopirox and tavaborole from the dorsal layer of the nail plate to the nail bed. In one embodiment, the pharmaceutically acceptable excipients to deliver the ciclopirox and tavaborole from the dorsal layer of the nail plate to the nail bed may be one or more of ethyl alcohol, isopropyl alcohol, propylene glycol, ethyl acetate, amyl acetate, and dimethyl sulfoxide.

In another aspect, the invention provides a topical pharmaceutical solution consisting of ciclopirox or salts thereof and a boron-containing antifungal agent or salts thereof as the antifungal agents and one or more pharmaceutically acceptable excipients to deliver the ciclopirox and tavaborole from the dorsal layer of the nail plate to the nail bed. In one embodiment, the pharmaceutically acceptable excipients to deliver the ciclopirox and tavaborole from the dorsal layer of the nail plate to the nail bed may be one or more of ethyl alcohol, isopropyl alcohol, propylene glycol, ethyl acetate, amyl acetate, and dimethyl sulfoxide.

In another aspect, the invention provides a topical pharmaceutical solution comprising (a) ciclopirox or salts thereof; (b) a boron-containing antifungal agent or salts thereof; and (c) a pharmaceutically acceptable excipient.

In another aspect, the invention provides a combination of ciclopirox, a boron-containing antifungal agent or salts thereof and one or more additional antifungal agents.

In another aspect, the invention provides a pharmaceutical composition comprising (a) tavaborole, (b) ciclopirox, and (c) a pharmaceutically acceptable excipient which exhibits excellent storage stability.

In another aspect, the invention provides a method of treating onychomycosis in a human, comprising administering to the human a therapeutically effective amount of ciclopirox and a boron-containing antifungal agent or salts thereof.

In another aspect, the invention provides a method for delivering tavaborole and ciclopirox from the dorsal layer of the nail plate to the nail bed. The method comprises contacting the cells of the dorsal layer of the nail plate with the combination of ciclopirox and a boron-containing antifungal agent or salts thereof capable of penetrating the nail plate, under conditions sufficient to penetrate the nail plate, and thereby delivering said combination.

In another aspect, the invention provides a method for treating onychomycosis in a patient suffering from the disease. The method involves topically administering a composition to at least one toe or fingernail of the patient, and the composition includes ciclopirox and a boron-containing antifungal agent or salts thereof.

Still other aspects and advantages of the invention will be apparent from the following detailed description of the invention.

### DETAILED DESCRIPTION OF THE INVENTION

The invention addresses the need for novel and effective combination therapy for onychomycosis. The present invention relates to combination therapies for the treatment of onychomycosis and related compositions. The compositions include ciclopirox and a boron-containing antifungal agent or salts thereof. Preferably, the boron-containing antifungal agent is tavaborole.

The inventors have further observed that the combination of ciclopirox and boron-containing antifungal agent acts synergistically, resulting in better penetration of both antifungal agents in the nail bed and treating a fungal infection, and improving the symptoms of the infection relatively faster than that achieved when the two antifungal agents are applied individually. The inventors have also observed that the antifungal effect of said combination is more than the combined antifungal effect of the individual antifungal agents.

The term “topical administration” refers to the application of a pharmaceutical agent to the external surface of the skin, nail, hair, claw or hoof of a human or animal, such that the agent crosses the external surface of the skin, nail, hair, claw or hoof and enters the underlying tissues. Topical administration includes application of the composition to intact skin, nail, hair, claw or hoof, or to a broken, raw or open wound of skin, nail, hair, claw or hoof. Topical administration of a pharmaceutical agent can result in a limited distribution of the agent to the skin and surrounding tissues or, when the agent is removed from the treatment area by the bloodstream, can result in systemic distribution of the agent.

‘MIC’, or ‘minimum inhibitory concentration’, is the point where compound stops more than 90% of cell growth relative to an untreated control.

The term “pharmaceutically acceptable excipients” refers to preservatives, antioxidants, fragrances, emulsifiers, dyes and excipients known or used in the field of drug formulation and that do not unduly interfere with the effectiveness of the biological activity of the active agent, and that are sufficiently non-toxic to the host or patient.

The abbreviations used herein generally have their conventional meaning within the chemical and biological arts.

In the present invention, the antifungal agents are, preferably, present in the free form, e.g., as an acid or base, rather than in the form of their salts. For example, ciclopirox may be used in the form of a free base or its olamine salt. “Ciclopirox” refers to 6-cyclohexyl-1-hydroxy-4methyl-2(1H)-pyridone and “Ciclopirox olamine” refers to the 2-aminoethanol salt of Ciclopirox.

Suitable boron-containing antifungal agents may be selected from compounds which are disclosed in U.S. Pat. No. 7,582,621 which is incorporated herein by reference. The preferred boron-containing antifungal agent is 5-fluoro-1, 3-dihydro-1-hydroxy-2, 1-benzoxaborole, which is also known as tavaborole.

In an embodiment, the invention provides a method of inhibiting the growth of a microorganism, or killing a microorganism, or both, comprising contacting the microorganism with ciclopirox and a boron-containing antifungal agent. Microorganisms are members selected from fungi, yeast, viruses, bacteria and parasites. In another exemplary embodiment, the microorganism is inside, or on the surface of an animal. In an embodiment, the animal is a human.

In another embodiment, the invention provides a method of treating or preventing an infection, or both. The method includes administering to the animal a therapeutically effective amount of ciclopirox and a boron-containing antifungal agent or salts thereof, sufficient to treat or prevent said infection. In another exemplary embodiment, the infection is a member selected from a systemic infection, a cutaneous infection, and an ungual or periungual infection.

In another embodiment, the invention provides a method of treating or preventing an ungual and/or periungual infection. The method includes administering to the animal a therapeutically effective amount of ciclopirox and a boron-containing antifungal agent or salts thereof, sufficient to treat or prevent said infection.

In another embodiment, the invention provides a method of treating or preventing onychomycosis. The method includes administering to the animal a therapeutically effective amount of a pharmaceutical formulation of the invention, sufficient to treat or prevent onychomycosis. In another exemplary embodiment, the method includes administering the pharmaceutical formulation of the invention at a site which is a member selected from the skin, nail, hair, hoof, claw and the skin surrounding the nail, hair, hoof and claw.

In another embodiment, the invention provides a method of delivering ciclopirox and a boron-containing antifungal agent or salts thereof from the dorsal layer of the nail plate to the nail bed. This method comprises contacting the cells of the dorsal layer of the nail plate with ciclopirox and a boron-containing antifungal agent or salts thereof capable of penetrating the nail plate, under conditions sufficient to penetrate the nail. The boron-containing antifungal agent has a molecular weight of between about 100 and about 200 Da. The boron-containing antifungal agent also has a log P value of between about 1.0 and about 2.6. The boron-containing antifungal agent additionally has a water solubility between about 0.1 mg/mL and 1 g/mL octanol/saturated water, thereby delivering said agent.

Also contemplated by the present invention is a boron-containing antifungal agent with a Log P value less than 2.5, a molecular weight less than 200 Da, and that is still able to penetrate the nail plate.

In one embodiment of the present invention the boron-containing antifungal agent has a water solubility between about 0.1 mg/mL to 1 g/mL in octanol saturated water. In one embodiment of the present invention the boron-containing antifungal agent has a water solubility of between 0.1 mg/mL and 100 mg/mL.

In another embodiment, the invention is a pharmaceutical composition which includes: (a) ciclopirox or salts thereof, (b) a boron-containing antifungal agent or salts thereof, and (c) a pharmaceutically acceptable excipient.

The composition may be a solution, cream, ointment, foam or spray, containing any concentration of ciclopirox and a boron-containing antifungal agent that produces a desirable pharmaceutical effect. Non-limiting examples of other ciclopirox or boron-containing antifungal agent concentrations include a 3% to 15% solution or cream. In an embodiment, ciclopirox and the boron-containing antifungal agent concentrations include 5%, 6%, 7%, 9%, 10%, 11%, 12%, and 13%.

Where a single additional antifungal agent is included in the composition, it is typically included at a concentration of 0.5%, 1%, 1.5%, 2%, 2.5%, 3%, 3.5%, 4%, 4.5% or 5% in the solution or cream.

Where two additional antifungal agents are included in the composition, their combined concentration is typically 0.5%, 1%, 1.5%, 2%, 2.5%, 3%, 3.5%, 4%, 4.5% or 5% in the solution or cream.

The additional antifungal agent which can be included in the combination of the present invention is typically selected from the following list: terbinafine; itraconazole; ketoconazole; fluconazole; derivatives of fluconazole; oxiconazole; sulconazole; clotrimazole; miconazole; econazole; azanidazole; bifonazole; butoconazole; chlormidazole; fenticonazole; imazalil; isoconazole; neticonazole; sertaconazole; tioconazole; naftifine; griseofulvin; amorolfine; sodium pyrithione, bifonazole/urea; and, propylene glycol-urea-lactic acid.

The pharmaceutical compositions of the invention can take a variety of forms adapted to the chosen route of administration. Those skilled in the art will recognize various synthetic methodologies that may be employed to prepare non-toxic pharmaceutical compositions incorporating the combination described herein. Those skilled in the art will recognize a wide variety of non-toxic pharmaceutically acceptable solvents that may be used to prepare solvates of the compounds of the invention, such as water, ethanol, propylene glycol, mineral oil, vegetable oil and dimethylsulfoxide (DMSO).

The compositions of the invention are administered topically in dosage unit formulations containing conventional non-toxic pharmaceutically acceptable carriers, adjuvants and vehicles. It is further understood that the best method of administration may be a combination of methods.

The compositions of the present invention comprises fluid or semi-solid vehicles that may include but are not limited to polymers, thickeners, buffers, neutralizers, chelating agents, preservatives, surfactants or emulsifiers, antioxidants, waxes or oils, emollients, sunscreens, and a solvent or mixed solvent system. The solvent or mixed solvent system is important to the formulation because it is primarily responsible for dissolving the drug. The best solvent or mixed solvent systems are also capable of maintaining clinically relevant levels of the drug in solution despite the addition of a poor solvent to the formulation. The topical compositions useful in the subject invention can be made into a wide variety of product types. These include, but are not limited to, lotions, creams, gels, sticks, sprays, ointments, pastes, foams, mousses, and cleansers. These product types can comprise several types of carrier systems including, but not limited to particles, nanoparticles, and liposomes. If desired, disintegrating agents can be added, such as cross-linked polyvinyl pyrrolidone, agar or alginic acid or a salt thereof such as sodium alginate. Techniques for formulation and administration can be found in Remington: The Science and Practice of Pharmacy, supra. The formulation can be selected to maximize delivery to a desired target site in the body.

Lotions, which are preparations that are to be applied to the skin, nail, hair, claw or hoof surface without friction, are typically liquid or semi-liquid preparations in which finely divided solid, waxy, or liquid are dispersed. Lotions will typically contain suspending agents to produce better dispersions as well as compounds useful for localizing and holding the active agent in contact with the skin, nail, hair, claw or hoof, e.g., methylcellulose, sodium carboxymethyl-cellulose, or the like.

Creams containing the active agent for delivery according to the present invention are viscous liquid or semisolid emulsions, either oil-in-water or water-in-oil. Cream bases are water-washable, and contain an oil phase, an emulsifier and an aqueous phase. The oil phase is generally comprised of petrolatum or a fatty alcohol, such as cetyl- or stearyl alcohol. The aqueous phase usually, although not necessarily, exceeds the oil phase in volume, and generally contains a humectant. The emulsifier in a cream formulation, as explained in Remington: The Science and Practice of Pharmacy, supra, is generally a nonionic, anionic, cationic or amphoteric surfactant.

Gel formulations can also be used in connection with the present invention. As will be appreciated by those working in the field of topical drug formulations, gels are semisolid. Single-phase gels contain organic macromolecules distributed substantially uniformly throughout the carrier liquid, which is typically aqueous, but also may be a solvent or solvent blend.

Ointments, which are semisolid preparations, are typically based on petrolatum or other petroleum derivatives. As will be appreciated by the ordinarily skilled artisan, the specific ointment base to be used is one that provides for optimum delivery for the active agent chosen for a given formulation and, preferably, provides for other desired characteristics as well, e.g., emolliency or the like. As with other carriers or vehicles, an ointment base should be inert, stable, non-irritating and non-sensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be had to Remington: The Science and Practice of Pharmacy, supra, for further information.

Liquid forms, such as lotions suitable for topical administration or suitable for cosmetic application, may include a suitable aqueous or nonaqueous vehicle with buffers, suspending and dispensing agents, thickeners, penetration enhancers, and the like. Solid forms such as creams, pastes and the like may include, for example, any of the following ingredients: water, oil, alcohol or grease as a substrate with surfactant, polymers such as polyethylene glycol, thickeners, solids and the like. Liquid or solid formulations may include enhanced delivery technologies such as liposomes, microsomes, microsponges and the like.

In an embodiment, the pharmaceutical composition is in the form of a simple solution. In an embodiment, the simple solution includes an alcohol. In a further embodiment, the simple solution includes alcohol and water. In a further embodiment, the alcohol is ethanol, ethylene glycol, propanol, polypropylene glycol, isopropanol or butanol. In another exemplary embodiment, the simple solution is a member selected from about 10% polypropylene glycol and about 90% ethanol; about 20% polypropylene glycol and about 80% ethanol; about 30% polypropylene glycol and about 70% ethanol; about 40% polypropylene glycol and about 60% ethanol; about 50% polypropylene glycol and about 50% ethanol; about 60% polypropylene glycol and about 40% ethanol; about 70% polypropylene glycol and about 30% ethanol; about 80% polypropylene glycol and about 20% ethanol; about 90% polypropylene glycol and about 10% ethanol.

Additives for topical formulations are well-known in the art, and may be added to the topical composition, as long as they are pharmaceutically acceptable and not deleterious to the epithelial cells or their function. Further, they should not cause deterioration in the stability of the composition. For example, the additive may be one or more of inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

The topical pharmaceutical compositions may also comprise suitable nail penetration enhancers. Examples of nail penetration enhancers include mercaptan compounds, acetyl cysteine, sulfites and bisulfites, keratolytic agents and surfactants. Nail penetration enhancers suitable for use in the invention are described in greater detail in Malhotra et al., J. Pharm. Sci., 91:2, 312-323 (2002), which is incorporated herein by reference in its entirety.

The topical pharmaceutical compositions may also comprise one or more suitable solvents. The ability of any solid substance (solute) to dissolve in any liquid substance (solvent) is dependent upon the physical properties of the solute and the solvent. When solutes and solvents have similar physical properties the solubility of the solute in the solvent will be the greatest. This gives rise to the traditional understanding that “like dissolves like”. Solvents can be characterized in one extreme as non-polar, lipophilic oils, while in the other extreme as polar, hydrophilic solvents. Oily solvents dissolve other non-polar substances by Van der Wals interactions while water and other hydrophilic solvents dissolve polar substances by ionic, dipole, or hydrogen bonding interactions. All solvents can be listed along a continuum from the least polar, i.e. hydrocarbons such as decane, to the most polar solvent being water. A solute will have its greatest solubility in solvents having equivalent polarity. Thus, for drugs having minimal solubility in water, less polar solvents will provide improved solubility with the solvent having polarity nearly equivalent to the solute providing maximum solubility. Most drugs have intermediate polarity, and thus experience maximum solubility in solvents such as propylene glycol or ethanol, which are significantly less polar than water. If the drug has greater solubility in propylene glycol (for example 8% (w/w)) than in water (for example 0.1% (w/w)), then addition of water to propylene glycol should decrease the maximum amount of drug solubility for the solvent mixture compared with pure propylene glycol. Addition of a poor solvent to an excellent solvent will decrease the maximum solubility for the blend compared with the maximum solubility in the excellent solvent.

### EXAMPLE 1

### Ciclopirox and Tavaborole Topical Solution

TABLE 1

| Sr. | | Formula 1 | Formula 2 | Formula 3 | Formula 4 |
|---|---|---|---|---|---|
| No. | Ingredients | (% w/w) | (% w/w) | (% w/w) | (% w/w) |
| 1 | Ciclopirox (base) | 4 | 6 | 8 | 10 |
| 2 | Tavaborole | 2 | 4 | 5 | 8 |
| 3 | Alcohol | 10 | 15 | 20 | 30 |
| 4 | Isopropyl Alcohol | 40 | 0 | 0 | 20 |
| 5 | Propylene Glycol | 17 | 33 | 10 | 10 |
| 6 | Ethyl Acetate | 5 | 10 | 15 | 20 |
| 7 | Amyl Acetate | 2 | 2 | 2 | 2 |
| 8 | Dimethyl | 20 | 30 | 40 | 0 |
| | Sulfoxide | | | | |
| | Total | 100 | 100 | 100 | 100 |

(Thickening agents and fragrances are optionally added).

Process: Alcohol, Isopropyl Alcohol, Propylene Glycol, Ethyl Acetate, Amyl acetate and Dimethyl Sulfoxide were mixed in a suitable stainless steel container fixed with Lighten' mixer until a clear solution was obtained. Ciclopirox was added to the solvent mixture until dissolved. Tavaborole was added to the solvent mixture and mixed well until dissolved and a clear solution was obtained.

In an alternate process, Ciclopirox and Tavaborole can be dissolved in Alcohol and Isopropyl Alcohol first, then the other ingredients may be added and dissolved until a clear solution is obtained.

1. A pharmaceutical composition comprising:
(a) ciclopirox or a salt thereof;
(b) a boron-containing antifungal agent or salts thereof; and
(c) one or more pharmaceutically acceptable excipients.

2. The composition of claim 1, wherein the salt of ciclopirox is ciclopirox olamine.

3. The composition of claim 1, wherein the boron-containing antifungal agent is tavaborole.

4. The composition of claim 1, wherein the composition further comprises one or more additional antifungal agents.

5. The composition of claim 4, wherein the one or more additional antifungal agents are selected from the group consisting of terbinafine, itraconazole, ketoconazole, fluconazole, derivatives of fluconazole, oxiconazole, sulconazole, clotrimazole, miconazole, econazole, azanidazole, bifonazole, butoconazole, chlormidazole, fenticonazole, imazalil, isoconazole, neticonazole, sertaconazole, tioconazole, naftifine, griseofulvin, amorolfine, sodium pyrithione, bifonazole/urea; and propylene glycol-urea-lactic acid.

6. The composition of claim 1, wherein the composition is in the form of solution, gel, cream, lotion, tincture, or ointment.

7. The composition of claim 1, wherein the one or more pharmaceutically acceptable excipients comprise one or more of ethyl alcohol, isopropyl alcohol, propylene glycol, ethyl acetate, amyl acetate, and dimethyl sulfoxide.

8. The composition of claim 7, wherein the composition consists essentially of ciclopirox and tavaborole as the active anti-fungal agents and ethyl alcohol, isopropyl alcohol, propylene glycol, ethyl acetate, amyl acetate, and dimethyl sulfoxide to deliver the ciclopirox and tavaborole from the dorsal layer of the nail plate to the nail bed.

9. The composition of claim 8, wherein the composition consists essentially of

| Ingredient | % (w/w) |
|---|---|
| Ciclopirox (base) | 4 |
| Tavaborole | 2 |
| Alcohol | 10 |
| Isopropyl Alcohol | 40 |
| Propylene Glycol | 17 |
| Ethyl Acetate | 5 |
| Amyl Acetate | 2 |
| Dimethyl Sulfoxide | 20 |

10. The composition of claim 7, wherein the composition consists essentially of ciclopirox and tavaborole as the active anti-fungal agents and ethyl alcohol, propylene glycol, ethyl acetate, amyl acetate, and dimethyl sulfoxide to deliver the ciclopirox and tavaborole from the dorsal layer of the nail plate to the nail bed.

11. The composition of claim 10, wherein the composition consists essentially of

| Ingredient | % (w/w) |
|---|---|
| Ciclopirox (base) | 6 |
| Tavaborole | 4 |
| Ethyl Alcohol | 15 |
| Propylene Glycol | 33 |
| Ethyl Acetate | 10 |
| Amyl Acetate | 2 |
| Dimethyl Sulfoxide | 30 |

12. The composition of claim 10, wherein the composition consists essentially of

| Ingredient | % (w/w) |
|---|---|
| Ciclopirox (base) | 8 |
| Tavaborole | 5 |
| Ethyl Alcohol | 20 |
| Propylene Glycol | 10 |
| Ethyl Acetate | 15 |
| Amyl Acetate | 2 |
| Dimethyl Sulfoxide | 40 |

13. The composition of claim 7, wherein the composition consists essentially of ciclopirox and tavaborole as the active anti-fungal agents and ethyl alcohol, isopropyl alcohol, propylene glycol, ethyl acetate, and amyl acetate to deliver the ciclopirox and tavaborole from the dorsal layer of the nail plate to the nail bed.

14. The composition of claim 13, wherein the composition consists essentially of

| Ingredient | % (w/w) |
|---|---|
| Ciclopirox (base) | 10 |
| Tavaborole | 8 |
| Ethyl Alcohol | 30 |
| Isopropyl Alcohol | 20 |
| Propylene Glycol | 10 |
| Ethyl Acetate | 20 |
| Amyl Acetate | 2 |

15. The composition of claim 7, wherein the composition consists of

| Ingredient | % (w/w) |
|---|---|
| Ciclopirox (base) | about 4-10 |
| Tavaborole | about 2-8 |
| Ethyl Alcohol | about 10-30 |
| Isopropyl Alcohol | about 0-40 |
| Propylene Glycol | about 10-33 |
| Ethyl Acetate | about 5-20 |
| Amyl Acetate | about 2 |
| Dimethyl Sulfoxide | about 0-40 |

16. A method of treating onychomycosis in a patient suffering from the disease comprises topically administering a composition comprising ciclopirox, a boron-containing antifungal agent, and one or more pharmaceutically acceptable excipients to at least one toe or fingernail of the patient.

17. The method of claim 16, wherein the composition comprises:

| Ingredient | % (w/w) |
|---|---|
| Ciclopirox (base) | about 4-10 |
| Tavaborole | about 2-8 |
| Ethyl Alcohol | about 10-30 |
| Isopropyl Alcohol | about 0-40 |
| Propylene Glycol | about 10-33 |
| Ethyl Acetate | about 5-20 |
| Amyl Acetate | about 2 |
| Dimethyl Sulfoxide | about 0-40 |

18. The method of claim 16, wherein the composition consists essentially of:

| Ingredient | % (w/w) |
|---|---|
| Ciclopirox (base) | about 4-10 |
| Tavaborole | about 2-8 |
| Ethyl Alcohol | about 10-30 |
| Isopropyl Alcohol | about 0-40 |
| Propylene Glycol | about 10-33 |
| Ethyl Acetate | about 5-20 |
| Amyl Acetate | about 2 |
| Dimethyl Sulfoxide | about 0-40 |

19. A method of delivering ciclopirox and a boron-containing antifungal agent or salts thereof from the dorsal layer of the nail plate to the nail bed, the method comprises contacting said cell with the combination of ciclopirox and a boron containing antifungal agent or salts thereof capable of penetrating the nail plate, under conditions sufficient to penetrate said nail plate, and thereby delivering said combination.

20. The method of claim 19, wherein boron-containing anti-fungal agent is tavaborole and the composition consists essentially of:

| Ingredient | % (w/w) |
|---|---|
| Ciclopirox (base) | about 4-10 |
| Tavaborole | about 2-8 |
| Ethyl Alcohol | about 10-30 |
| Isopropyl Alcohol | about 0-40 |
| Propylene Glycol | about 10-33 |
| Ethyl Acetate | about 5-20 |
| Amyl Acetate | about 2 |
| Dimethyl Sulfoxide | about 0-40 |
